(19) United States
(12) Patent Application Publication
(10) Pub. No.: US 2021/0369161 A1
(43) Pub. Date: Dec. 2, 2021

(54) SYSTEM AND METHOD FOR DETECTION AND CONTINUOUS MONITORING OF NEUROLOGICAL CONDITION OF A USER

(71) Applicant: Antonio Visconti, Menlo Park, CA (US)

(72) Inventor: Antonio Visconti, Menlo Park, CA (US)

(21) Appl. No.: 17/318,688

(22) Filed: May 12, 2021

Related U.S. Application Data
(60) Provisional application No. 63/033,515, filed on Jun. 2, 2020.

(51) Int. Cl. A61B 5/16; A61B 3/11; A61B 3/14; A61B 3/00; A61B 5/00; A61B 5/1171; A61B 5/18; G06K 9/00; B60R 11/04

(52) U.S. Cl. CPC A61B 5/163; A61B 3/112; A61B 3/14; A61B 3/0033; A61B 5/4845; A61B 5/6803; B60R 2011/0003; A61B 5/18; A61B 5/6893; A61B 5/0002; G06K 9/0061; G06K 9/00617; B60R 11/04; A61B 5/1171

(57) ABSTRACT

A system and method are provided for detecting and continuously monitoring Pupillary Light Reflex responses and neurological conditions to determine probability and degree of impairment to a user due to intoxication or neurological disorder. In one embodiment, the system includes a camera in a housing affixed to an article worn on the head of a user operable to enable the camera to view at least one eye of the user, a portable video capture device (VCD) wired or wirelessly coupled, or integrated to the camera to receive video therefrom, and software executed by a processor in the VCD. The software includes modules to locate and measure a change in a feature of the eye, extract data therefrom and predict a degree of impairment of the user, and output the probability and degree of impairment to the user and/or to a third party monitor. Other embodiments are also disclosed.

### CROSS-REFERENCE TO RELATED APPLICATIONS

This application claims the benefit of priority under 35 U.S.C. 119(e) to U.S. Provisional Patent Application Ser. No. 63/033,515, filed Jun. 2, 2020, which is incorporated by reference herein in its entirety.

### TECHNICAL FIELD

The present disclosure relates generally to a system and method for detection of neurological condition of a user, and more particularly to a method, system and application or software program designed to detect neurological condition, including impairment due to fatigue, sleep deprivation, ingesting of drugs, alcohol and/or neurological disorders by automatically and continuously monitoring an individual Pupillary Light Reflex (PLR) while carrying out any activity like driving, walking, working or simply looking around.

### BACKGROUND

The pupillary light reflex (PLR) is a reflex that controls the diameter of the pupil, in response to the intensity of light that falls on the retinal ganglion cells of the retina in the back of the eye, thereby assisting in adaptation to various levels of lightness/darkness. A greater intensity of light causes the pupil to constrict, whereas a lower intensity of light causes the pupil to dilate. Thus, the pupillary light reflex regulates the intensity of light entering the eye. By simply looking at brighter or darker objects or looking in the direction or away from light sources, the PLR continuously adjusts the size of the pupil. It is well documented in the medical and scientific community that an individual PLR may be altered by fatigue, sleep deprivation, ingesting or otherwise introducing an intoxicating substance, such as alcohol or a drug, or by a medical condition such as a concussion or by other neurological disorders such as diabetic neuropathy. Thus, the analysis of the PLR can be used to detect impairment due to intoxicating substances and/or all neurological disorder affecting an individual PLR response.

Impairment can be brought about by fatigue, sleep deprivation or as a result of ingesting or otherwise introducing an intoxicating substance, such as alcohol or a drug. Impairment can also be brought about by a neurological disorder caused by a medical condition such as a concussion or by other pathologies like stroke, brain injury and diabetic neuropathies. By impairment it is meant a diminution of a speed or quality in mental and motor functions of the effected individual. Impairment can include or result in loss or diminishment in judgment, self-control, reasoning, memory, speech and/or coordination. By neurological disorder is meant any disorder of the nervous system.

Extreme impairment is readily recognizable to others, and, generally, to the individual—although because judgment is impaired the individual may not recognize or acknowledge the impairment. More problematic are situations in which the individual is only mildly impaired and thus may not be aware of any impairment at all. For example, because of a multitude of factors that affect blood alcohol concentration, i.e., age, gender, rate of consumption, body mass, food consumption and alcohol intolerance common among some ethnic groups, it is very difficult for an individual to assess his or her own impairment. While earlier stages of alcohol impairment may be undetectable to the drinker and others, it is known even small amounts of alcohol may affect one's ability to drive, and a person will possibly be too impaired to drive or perform a dangerous task before appearing or maybe even feeling “drunk” or “high.”

The same situation can arise when a person has suffered a blow to the head and have a concussion or is suffering from extreme fatigue or sleep deprivation, but insist that they ‘feel fine,’ and do not require medical attention or rest.

Chronic illnesses like diabetes are known to cause diabetic neuropathies that alter the PLR response over time. People with diabetes can, over time, develop nerve damage throughout the body. The change is subtle and slow and some people with nerve damage have no symptoms.

Thus, there is a need for an easy to use, portable and ubiquitous system and method to permit detection and continuous monitoring of a person to measure a degree of impairment due to the consumption of impairing substances and/or physical or neurological conditions for safety purposes. There is a further need for the system to operate in such a way as not to impede tasks or functions performed by the person being monitored.

### SUMMARY

The objective of the invention is to provide a non-invasive way to measure and continuously monitor Pupillary Light Reflex (PLR) response test and other involuntary eye movements and correlate such measurement to an impairment level that can be associated with fatigue, alcohol, drug consumption, trauma and/or other neurological disorder due to any possible cause that affects the individual PLR response. An important aspect of the invention is the implementation in such way as to enable regular and substantially continuous monitoring of the neurological condition of the individual in a non-intrusive or minimally intrusive manner.

In one embodiment, the system includes a camera in a housing affixed to an article worn on the head of a user configured or operable to enable the camera to view at least one eye of the user, a portable video capture device (VCD) wired or wirelessly coupled to the camera to receive video therefrom, and software executed by a processor in the VCD. Generally, the software includes a video capture module to continuously or repeatedly capture a number of images or video of the eye using the camera, a local correlation and prediction module to predict a degree of impairment to the user, generate alerts messages or signals, and a user interface module to output the probability and degree of impairment to the user, and, optionally, to a third party monitor. More specifically, the local correlation and prediction module includes program code to: locate a feature of the eye; measure a change in the feature relative to a stored image, or over a predetermined time; extract data from the measured change in the feature; calculate a number of parameters from the extracted data; and correlate the calculated parameters with predetermined reference parameters and predict a degree of impairment based on the results of the correlation.

### BRIEF DESCRIPTION OF THE DRAWINGS

Embodiments of the present invention will be understood more fully from the detailed description that follows and from the accompanying drawings and the appended claims provided below, where:

FIG. 1A through 1D illustrate eye feature(s) measured, the effect of a light source on the eye feature(s), and the effect of movement on eye feature(s) captured according to embodiments of the present disclosure;

FIG. 2A is a graph of a normal, unimpaired reaction to a high intensity light stimuli showing pupillary constriction over time following exposure to high intensity light according to embodiments of the present disclosure;

FIG. 2B is a graph showing a test of pupillary constriction over time following exposure to a high intensity light stimuli as compared to a reference graph developed from a reference data set according to embodiments of the present disclosure;

FIG. 2C is a graph of pupillary movements over a longer period of time in which an individual is exposed to continuous light variations

FIG. 3 illustrates a block diagram of a system according to an embodiment of the present disclosure;

FIG. 4 is a flowchart illustrating a method to continuously monitor Pupillary Light Reflex (PLR) responses to determine probability and degree of impairment to a user according to an embodiment of the present disclosure;

FIG. 5 illustrates an embodiment of a system in which the camera is in a housing affixed to glasses or goggles to be worn on the head of a user and the video capture device (VCD) is a portable video capture device (VCD) included within a portable electronic device;

FIG. 6 illustrate an embodiment of a system in which the video capture device includes a camera in a housing affixed to a helmet worn on the head of a user;

FIG. 7 illustrate an embodiment of a system in which the video capture device includes a camera in a housing affixed to a headset worn on the head of a user;

FIG. 8 illustrate an embodiment of a system in which the camera is in a housing affixed to a surface in a vehicle operated by a user;

FIG. 9 is a flowchart illustrating a method to perform video processing according to an embodiment of the present disclosure;

FIG. 10 is a flowchart illustrating a method to perform eye feature(s) measurements according to an embodiment of the present disclosure;

FIG. 11 is a flowchart illustrating a method to perform correlation and prediction process according to an embodiment of the present disclosure;

FIG. 12 is a flowchart illustrating a method to perform user identification according to an embodiment of the present disclosure;

FIG. 13 is a flowchart illustrating a method to perform user authorization according to an embodiment of the present disclosure; and

FIGS. 14A and 14B is a flowchart illustrating a method to continuously monitor PLR responses to determine a probability and degree of impairment to a user using a system including a camera affixed to an article worn by the user according to an embodiment of the present disclosure.

### DETAILED DESCRIPTION

The present disclosure is directed generally to a system and method for testing and automatically and continuously monitoring impairment of an individual due to the influence of alcohol, drugs, an injury, fatigue and/or or other neurological disorder due to any possible cause that affects an individual's pupillary light reflex (PLR) response. The invention may be utilized to detect impairment due to the consumption of impairing substances like alcohol, marijuana or other drugs, early sign of potential neurological damage following a trauma event like a concussion or monitor the progress of a neurological disorder like a diabetic neuropathy.

In the following description, for purposes of explanation, numerous specific details are set forth in order to provide a thorough understanding of the present invention. It will be evident, however, to one skilled in the art that the present invention may be practiced without these specific details. In other instances, well-known structures, and techniques are not shown in detail or are shown in block diagram form in order to avoid unnecessarily obscuring an understanding of this description.

Reference in the description to “one embodiment” or “an embodiment” means that a particular feature, structure, or characteristic described in connection with the embodiment is included in at least one embodiment of the invention. The appearances of the phrase “in one embodiment” in various places in the specification do not necessarily all refer to the same embodiment. The term to couple as used herein may include both to directly electrically connect two or more components or elements and to indirectly connect through one or more intervening components.

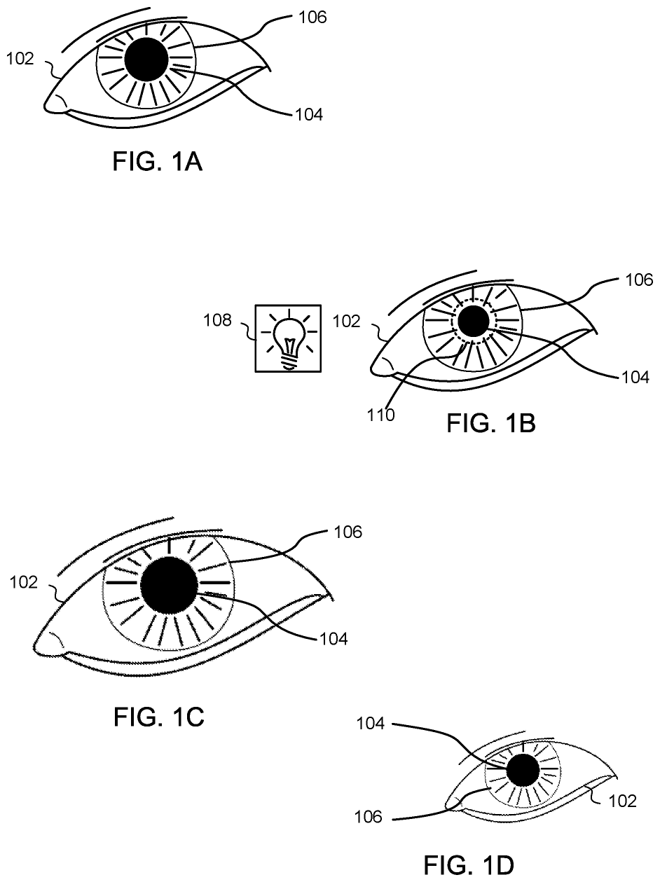

FIGS. 1A through 1D illustrate eye feature(s) measured, the effect of a light source on the eye feature(s), and methods to accurately measure change in pupil dimension in a video recording due to light stimuli while compensating measurement error due to movement during video capture.

FIG. 1A shows an eye 102 including a pupil 104 and an iris 106 prior to exposure to light stimuli. It is noted that the iris 106, unlike the pupil 104 is not affected by light. FIG. 1B shows an eye 102 including a pupil 104 and an iris 106 after exposure to light stimuli from a light source 108. It is noted that the pupil 104 contracted from an initial size represented by dashed circle 110.

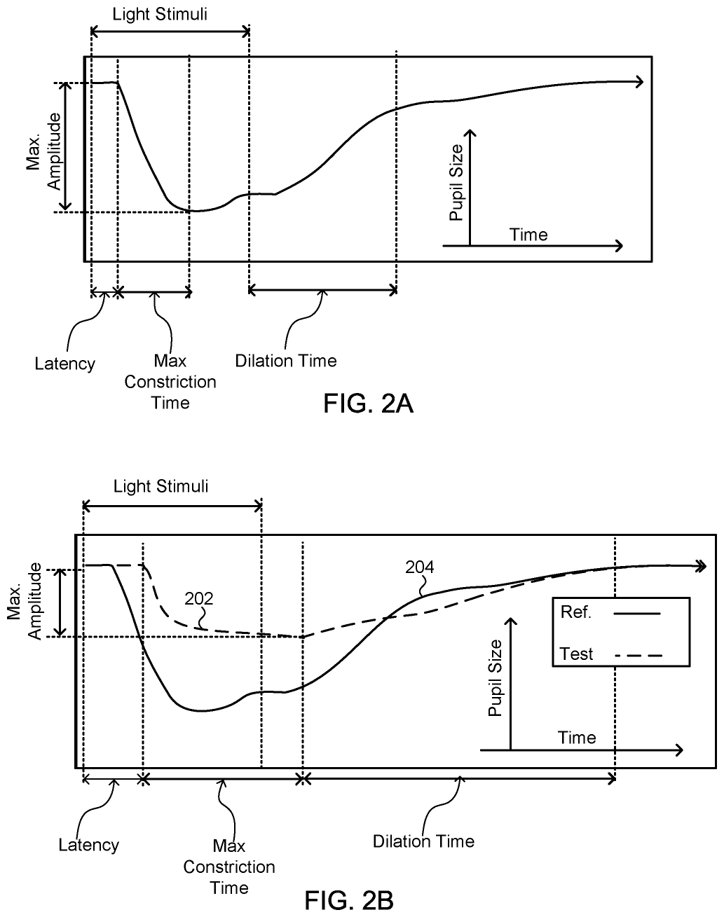

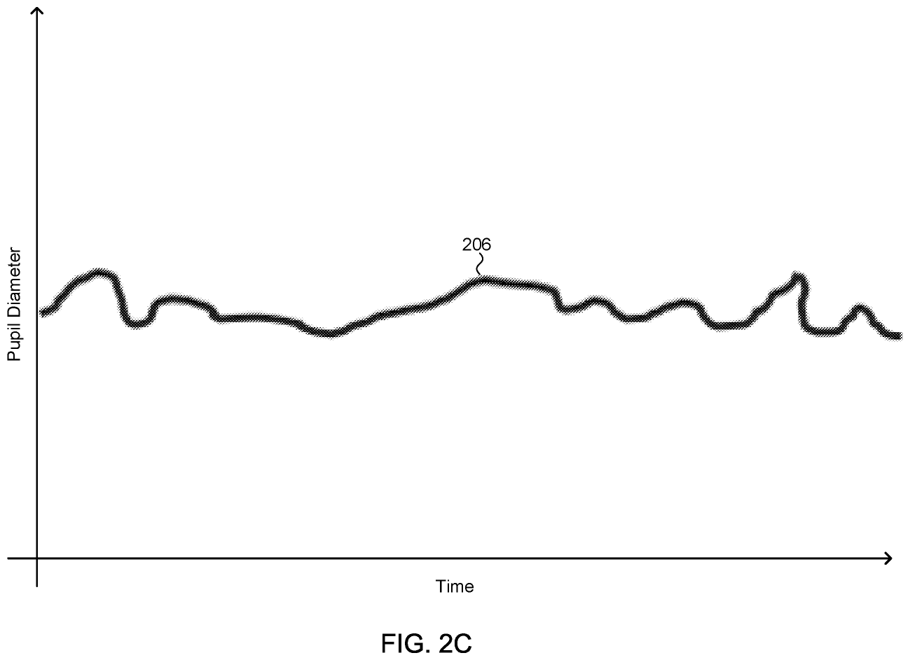

FIGS. 1C and 1D shows the effect of camera movement towards or away from the eye on pupil size and an iris 106 in captured video frames. Referring to FIG. 1C it is noted as the camera moves closer to the eye both the pupil 104 and iris 106 get larger. FIG. 1D shows that as the camera moves away from the eye both the pupil 104 and iris 106 get smaller. Thus, in prior art approaches to measuring pupil constriction or dilation the camera was required to be fixed relative to the eye. In contrast, the method using the system of the present disclosure, measures the pupil size relative to an outer edge of the iris, is therefore tolerant of movement during the predetermined time in which video is captured. The described process allows the acquisition of a PLR response signal, such as those shown in FIGS. 2A, 2B and 2C, that is independent from the camera movement, and which can be mathematical analyzed and described by calculating key parameter in the time and frequency domains.

Data analysis is the process of extracting critical information from the PLR response signal created by the eye features measurement process. The data analysis performed will now be described with reference to FIGS. 2A, 2B and 2C.

FIG. 2A is a graph of a normal reaction to a high intensity light stimuli consisting of a pupillary constriction of pupil size over time following exposure to a high intensity light. Referring to FIG. 2A, it is shown that a constriction in pupil size reaches a maximum amplitude following a brief latency period a maximum constriction time, that is the time required for a particular individual's pupil to constrict following exposure to a high intensity light stimuli when unimpaired. Dilation time is the time required for the pupil to return to the previous size following removal of the light stimuli. It is noted that latency, maximum constriction time, and dilation time for an unimpaired individual can vary from individual to individual due to age, health status, state of consciousness, etcetera, however all will generally fall within range of range of times. Thus, in some embodiments the system and method of the present disclosure can use a baseline or reference graph of a pupillary constriction following exposure to a high intensity light stimuli. In other, preferred embodiments a reference data set is established and stored for an individual being monitored at a time when the individual is known to be unimpaired. Generally, the reference data set can include are calculating or measuring and storing several parameters in the time domain, such as a maximum amplitude, latency, maximum constriction time, dilation time, and other parameters, like spectral power, frequency response that are extracted from a spectral analysis in the frequency domain of the PLR response signal for a normal, unimpaired response for the individual.

FIG. 2B is a graph showing a test of pupillary constriction over time following exposure to a constant high intensity light stimuli as compared to a reference graph developed from a reference data set. Dashed line 202 represents the graph of the test and line 204 represents the reference graph. Referring to dashed line 202 it is noted that latency, maximum constriction time, and dilation time have all increased substantially, with latency and maximum constriction time nearly doubling, and the maximum amplitude occurring after removal of the light stimuli. It is further noted that the maximum amplitude of the pupil constriction is substantially reduced.

FIG. 2C is a graph of pupillary movements over a longer period of time in which an individual is exposed to continuous light variations, rather than a single PLR measurement that is artificially induced with a sudden constant light stimulus. Line 206 represents changes in pupil diameter over time due to the continuous light variations, which can be compared to a predefined or predetermined maximum permissible changes in pupil size (amplitude) over a predetermined time, such that changes greater than the predetermined maximum requiring a longer time than the predetermined time for the pupil size to recover, would indicate a substantial probability of impairment. Referring to FIG. 2C, an individual in movement is continuously exposed to different light conditions and the pupil size is constantly adjusting, and changing size over time, such as while a person is looking around while working or operating a vehicle. Several parameters, in the time domain, are calculated and stored, like amplitude, speed of constriction or dilation, pupil size oscillations and other parameters that are extracted from a spectral analysis in the frequency domain of the pupillary response and variation of current test data against a Reference Data Set. This means analyzing the frequency response of the brain/ocular system to detect anomalies compared with the same analysis performed on the individual when impaired.

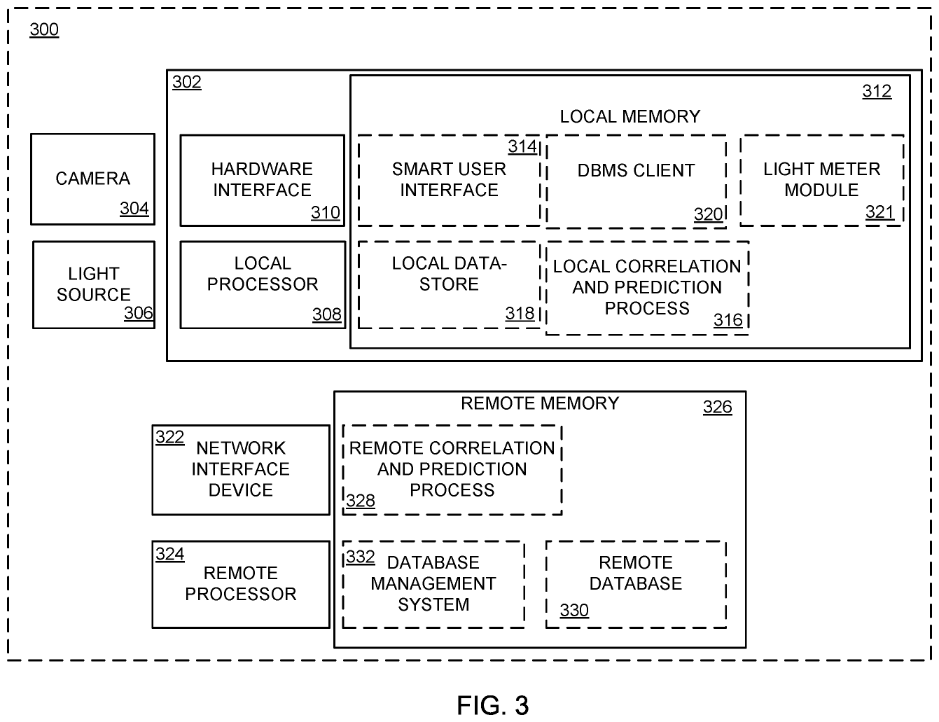

FIG. 3 illustrates a block diagram of a system 300 for performing an impairment test according to an embodiment of the present disclosure. Referring to FIG. 3 the system 300 generally includes a video capture device (VCD 302), a still or video camera 304, and, optionally, a light source 306 that can be controlled by the VCD to provide light stimuli for testing the pupillary light reflex (PLR) response. It is noted that in some embodiments the light source 306 is not necessary as the changing conditions of ambient or environmental light detected by the system 300 provide sufficient light stimuli for the PLR response testing. It is further noted that in some embodiments the VCD 302 is a portable VCD (PVCD) embodied in a portable electronic device, such as a cellular telephone. In some of these embodiments, the camera 304 and/or light source 306 are also embodied in the portable electronic device. In other embodiments the camera 304 and/or light source 306 are housed in one or more separate housings and coupled to the VCD 302 through either or a wired or wireless interface. The VCD 302 further includes a local processor 308, a hardware interface 310, and a local memory 312. The camera 304 is configured or operable to continuously or repeatedly capture a number of images or video of the eye over a predetermined time to determine a probability and degree of impairment to a user using a system. The local processor 308 is configured or operable to execute a software program or application to locate and measure a change in a feature of the eye over the predetermined time, analyzing the changes and extracting data therefrom, calculating a number of parameters from the extracted data, and correlating the calculated parameters with predetermined reference parameters to predict a probability and degree of impairment. The hardware interface 310 can include a display and/or auditory device, to communicate to a user the probability and degree of impairment.

The local memory 312 can store software (SW) including user interface SW 314, local correlation and prediction process SW 316, a local data-store 318, data-store management system (DBMS) client SW 320 and light meter software or module (LMM) 321. The user interface SW 314 includes computer program code to communicate with the user via the hardware interface. The local correlation and prediction process SW 316 includes computer program code executed by the processor to locate and measure a change in a feature of the eye, analyze and extract data from the changes, and calculate and correlate a number of parameters with predetermined reference parameters to predict a probability and degree of impairment. The local data-store 318 includes computer program code to store and retrieve information necessary to perform the impairment test, including predetermined reference parameters and, optionally user data on the person undergoing test. The DBMS client SW 320 includes computer program code to update or managing the local data-store 318 with customized parameters used by the correlation and prediction process to calculate the resultant probability and degree of impairment of the person following the correlation and prediction step and store and maintain historical measurement data. The light meter module 321 includes computer program code to direct the user to reduce the impact of environmental or ambient light improving the video capture.

Optionally or preferably in some embodiments, such as that shown, the VCD 302 is a network enabled device and the system 300 further includes a network interface device 322, that connects to a cellular telephone tower or a wireless access point, through which the network enabled VCD can be coupled to a remote processor 324 and/or a remote server or remote memory 326. Like the local processor 308, the remote processor 324 can be configured or operable to execute one or more software programs including programs to locate and measure a change in a feature of the eye over the predetermined time, analyze the changes and extract data therefrom, calculate a number of parameters from the extracted data, and correlate the calculated parameters with predetermined reference parameters to predict a probability and degree of impairment.

The remote memory 326 can store software (SW) including remote correlation and prediction process SW 328, a remote data-store 330, and data-store management system (DBMS) SW 332. The remote correlation and prediction process SW 328 includes computer program code executed by the processor to locate and measure a change in a feature of the eye, analyze and extract data from the changes, and calculate and correlate a number of parameters with predetermined reference parameters to predict a probability and degree of impairment. The remote data-store 330 includes computer program code to store and retrieve information necessary to perform the impairment test, including predetermined reference parameters and, optionally user data on the person undergoing test. The DBMS SW 332 includes computer program code to update or managing the remote data-store 330 with the resultant probability and degree of impairment of the person following the correlation and prediction step. It will be understood that the remote processor 324 and the remote DBMS SW 332 can be desirably used to maintain and update data of all users of the system for the purpose of analyzing measurements and results over the large user base. The data is used for a continuous refinement of the correlation and prediction process.

Suitable VCDs for use with the system and method of the present disclosure may include any portable, electronic device either including or capable of coupling to and receiving data from a camera and recording the resulting images or video capture. Preferably, the VCD further includes a user interface, a processor and is network enabled. A suitable VCD can include, for example, a smartphone, a portable computer, personal digital assistant, a digital camera, or a tablet computer.

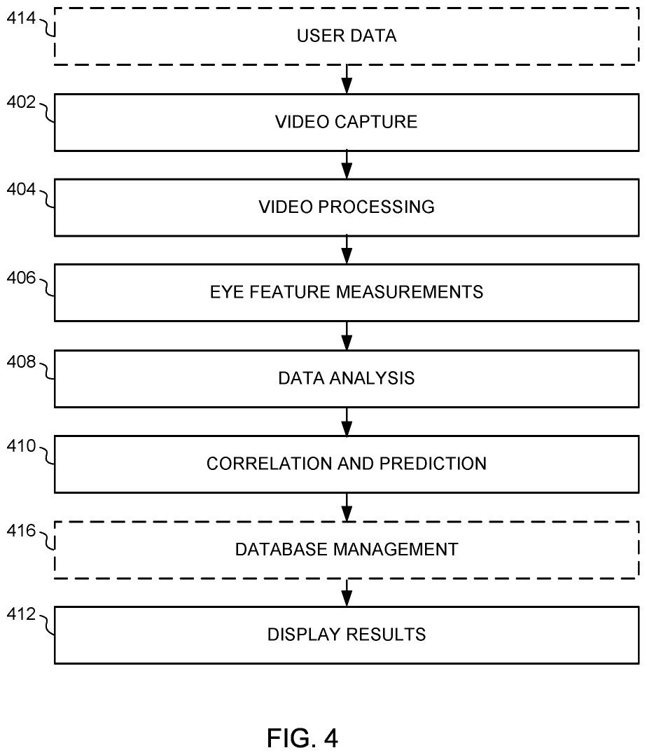

FIG. 4 is a flowchart illustrating the most general embodiment of a method to continuously or repeatedly over a short interval of time automatically monitor PLR responses to determine probability and degree of impairment to a user due to intoxication or neurological disorder.

Referring to FIG. 4, the method begins with capturing video or multiple images of an eye exposed to light stimuli over a predetermined time using a camera of a portable video capture device or VCD (step 402). As noted above, the light stimuli may originate from a light source in the system positioned near the eye of the user or individual being monitored and controlled by the VCD, or from environmental light originating from a light source external to the system. Next, the captured video is processed to locate a feature of the eye (step 404). Features of the eye can include a pupil, an iris and/or a border between the pupil and the iris or any other discernible feature. A change in the located feature in response to the light stimuli over the predetermined time is measured (step 406). Next, data extracted from the measured change in the feature is analyzed (step 408). The data analysis can include calculating a number of key descriptive parameters, in the time and frequency domains, from the PLR response signal. Next, the calculated parameters are correlated with predetermined reference parameters in a data-store and a probability and degree of impairment predicted based on the results of the correlation (step 410). Finally, the resultant probability and degree of impairment of the person, is output through a user interface in the VCD, such as a display and/or auditory interface to a user (step 412). It is noted that user may be the person undergoing the impairment test or another individual.

Optionally, as shown in FIG. 4, the method may further include an initial step of receiving data, such as a reference data set, on the person undergoing the impairment test (step 414), and updating or managing the data-store (step 416) with the resultant probability and degree of impairment of the person following the correlation and prediction step (step 410). User data can also include a name of the person undergoing the impairment test, contact information, age, gender, height, weight, body mass, ethnicity, and other information required for the correlation and prediction step. The data-store may include a local data-store stored in a local memory of the VCD, and/or a remote data-store stored in a remote memory coupled through a network to a network enabled VCD.

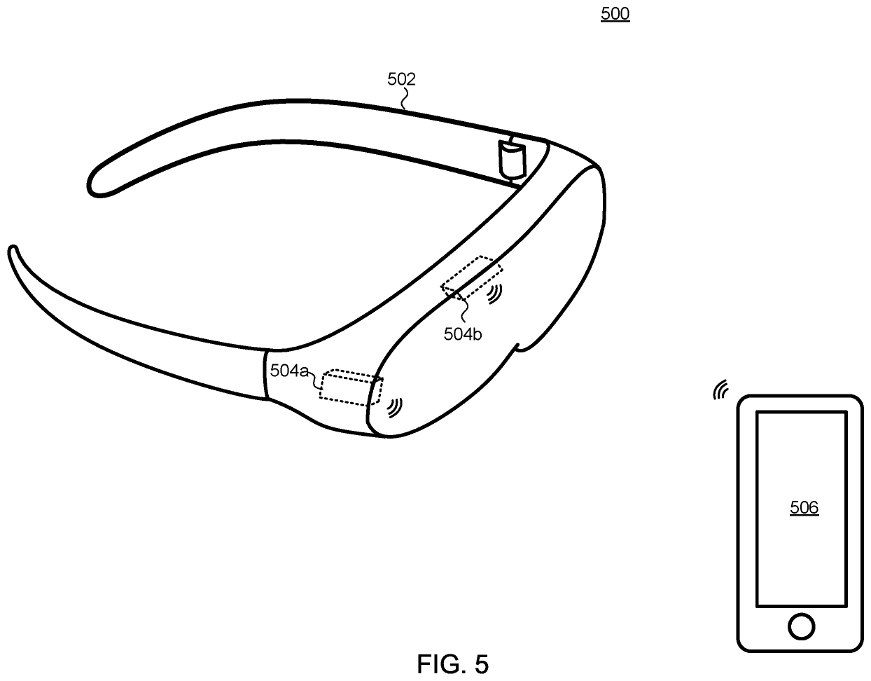

FIG. 5 illustrates an embodiment of a system 500 in which the article 502 to which on or more housings 504a, 504b, each enclosing a camera (not shown separately in this figure), and, optionally a light source or LMM to measure ambient light, is affixed includes glasses or goggles, such as safety glasses or goggles, worn on the head of a user and the portable video capture device (VCD 506) includes a portable electronic device, such as a cellular telephone. It is noted that the housings 504a, 504b, enclosing the cameras can be removably or fixedly attached to the glasses or goggles (article 502) at any position affording an unobstructed view of at least one eye of the user, without impeding the user's vision or causing undue discomfort. For example in the embodiment shown one housing, housing 504a, is affixed to the article 502 proximal to a side arm of the glasses or goggles, while another (housing 504b) is affixed elsewhere on the body of the glasses or goggles, such as a point near the bridge thereof. It is noted that while multiple cameras enclosed in multiple housings 504a, 504b, are shown in FIG. 5, a single camera enclosed in a single housing, either 504a or 504b, is sufficient to practice the system and method of the present invention. It is further noted that in some applications it may be desirable to have multiple cameras enclosed in multiple housings 504a, 504b, each positioned and configured or operable to view a separate eye of the user. This embodiment including multiple cameras can be particularly useful in detecting impairment due to certain medical conditions, such as a stroke, in which a pupil in one eye reacts differently to light stimuli.

In some embodiments, such as that shown, the camera is independently powered by a battery or other power source in the housing or article 502 to which it is attached, and the camera is wirelessly coupled to the VCD 506, for example through a Bluetooth or other RF connection. Alternatively, the camera in the housing 504a, 504b, can be coupled by wire or a wire connection to the VCD 506 to both power the camera and transmit signals for image or video to the VCD.

In other embodiments, the housing 504a, 504b, for the camera can further include a fully functional VCD, with all the processing and communication capabilities.

Optionally, as noted above and with respect to FIG. 3, in some embodiments the system 500 can further include a light source (not shown in this figure) controlled by the VCD 506 to provide light stimuli for the PLR response test. The light source can be enclosed within the same housing 504a, 504b, as the camera(s), or can be separately affixed to the article 502 either directly or enclosed in a separate housing. As with the housing 504a, 504b, for the cameras the light source or the housing therefore can be removably or fixedly attached to the glasses or goggles (article 502) at any position affording an unobstructed illumination of at least one eye of the user.

Finally, as noted above with respect to FIG. 3 suitable VCDs 506 for use with the system 500 and method of the present disclosure may include any portable, electronics device capable of coupling to and receiving data from the camera and recording the resulting images or video capture, and a user interface, including, for example, a smartphone, a portable computer, personal digital assistant, a digital camera, or a tablet computer. Preferably, the VCD further includes a processor and is network enabled. The VCD can be also fully integrated with the camera housing.

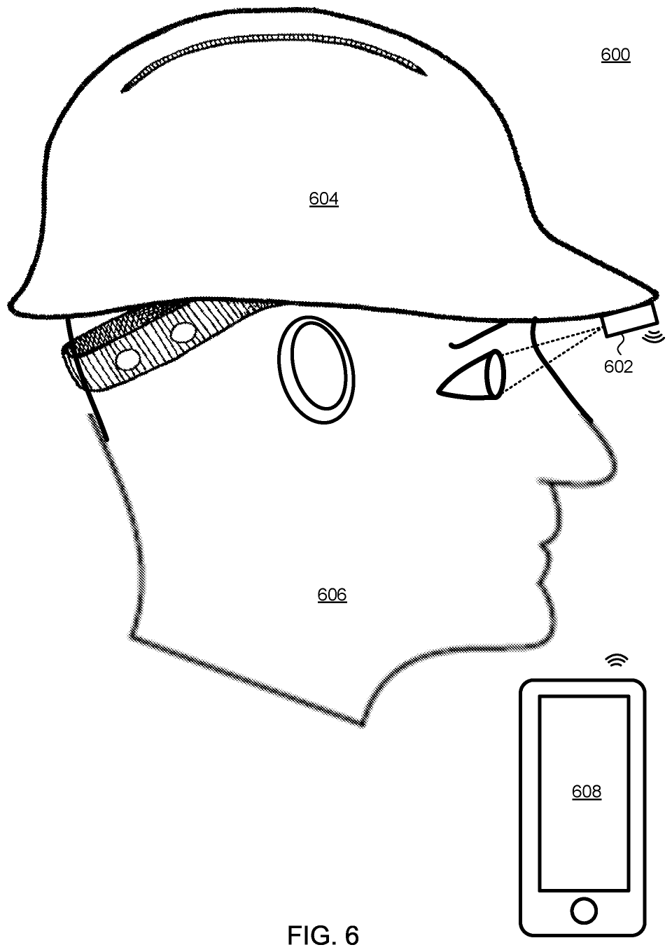

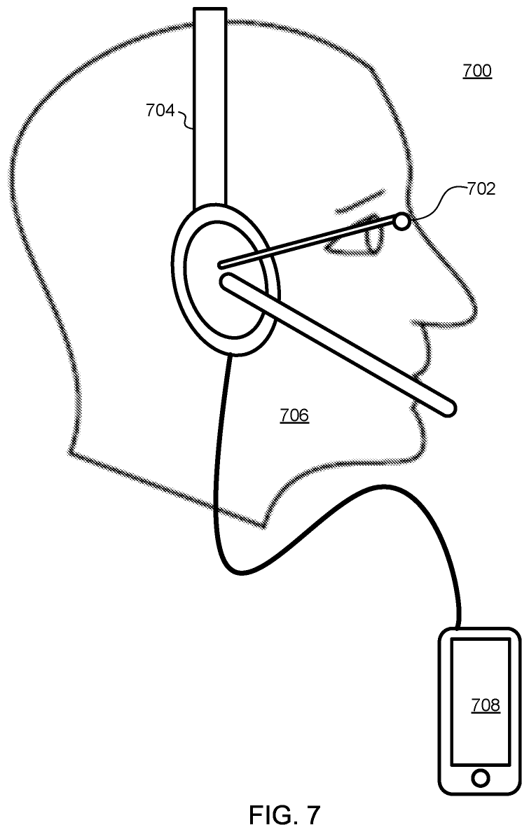

Another embodiment of a system 600 includes a camera in a housing 602 affixed to a protective head covering 604 worn on the head of a user 606 is shown in FIG. 6. The Referring to FIG. 6 the protective head covering 604 can include a hard or soft hat, or a helmet, such as used in safety equipment for sports, games or occupations, such as when operating equipment or motorized vehicles. As in the embodiments described above with respect to FIGS. 3 and 5, the system 600 further includes a VCD 608 that is wirelessly coupled to the camera enclosed in the housing 602 (as shown), or coupled through a wired connection (not shown).

Again it is noted that while a single camera enclosed in a single housing 602 is shown in FIG. 6, in some applications it may be desirable to have multiple cameras enclosed in multiple housings, each positioned and configured or operable to view a separate eye of the user. The housing 602, enclosing the camera can be removably or fixedly attached to the article (protective head covering 604) at any position affording an unobstructed view of at least one eye of the user, without impeding the user's vision or causing undue discomfort.

Optionally, in some embodiments the system 600 can further include a light source (not shown in this figure) controlled by the VCD 608 to provide light stimuli for the PLR response test. The light source can be enclosed within the same housing 602 as the camera, or can be separately affixed to the article (protective head covering 604) either directly or enclosed in a separate housing at any position affording an unobstructed illumination of at least one eye of the user.

In yet another embodiment, shown in FIG. 7 the system 700 includes a camera in a housing 702 affixed to a frame or head-band, such as a headset 704 or face-shield worn on the head of a user 706, and coupled to a VCD 708. In the embodiment shown, the VCD 708 is coupled through a wired connection to the camera enclosed in the housing 702.

As with the embodiments described above the system 700 can include one or more cameras in one or more housings 702 removably or fixedly attached to the headset 704.

Optionally, the system 700 can further include a light source in the housing 702 (not shown separately in this figure), and controlled by the VCD 708 to provide light stimuli for the PLR response test. The light source can be enclosed within the same housing 702 as the camera, or can be separately affixed to the headset 704 either directly or enclosed in a separate housing at any position affording an unobstructed illumination of at least one eye of the user.

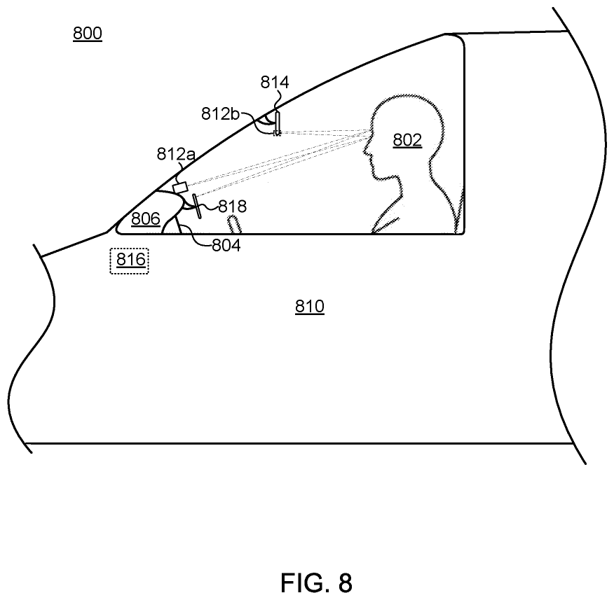

In yet another embodiment, shown in FIG. 8 the system 800 includes one or more cameras in one or more housings in or affixed to a surface of a machine being operated by a user 802. The housing is positioned facing a face of the user so that the camera is able to view at least one eye of the user 802. The surface can include a surface, such as an instrument panel 804 or dashboard 806, in a vehicle 810 being operated by the user. The housing can include a housing 812a either fixedly or removably attached to the surface, shown here as a dashboard in an airplane, train, automobile or watercraft, or can be a housing 812b included in a mirror 814 of the vehicle. In some embodiments, such as where the housing 812b is included in the mirror 814 or behind an instrument panel 804 (not shown in this figure), the surface is substantially transparent to at least some wavelengths of light to which the camera is sensitive, and the housing with the camera housed therein is affixed to an opposite side of the surface from the user.

The system 800 further includes a video capture device (VCD 816) and coupled to the camera and/or light source (not shown separately in this figure) through either or a wired or wireless interface. As with the embodiments described above the VCD 816 includes a local processor, a hardware interface, and a local memory, and the camera is configured or operable to continuously or repeatedly capture a number of images or video of the eye over a predetermined time to determine a probability and degree of impairment to a user using a system.

In another version of this embodiment the video capture device is a portable VCD (PVCD), such as a cellular telephone 818, and the camera and/or light source are embodied in the cellular telephone.

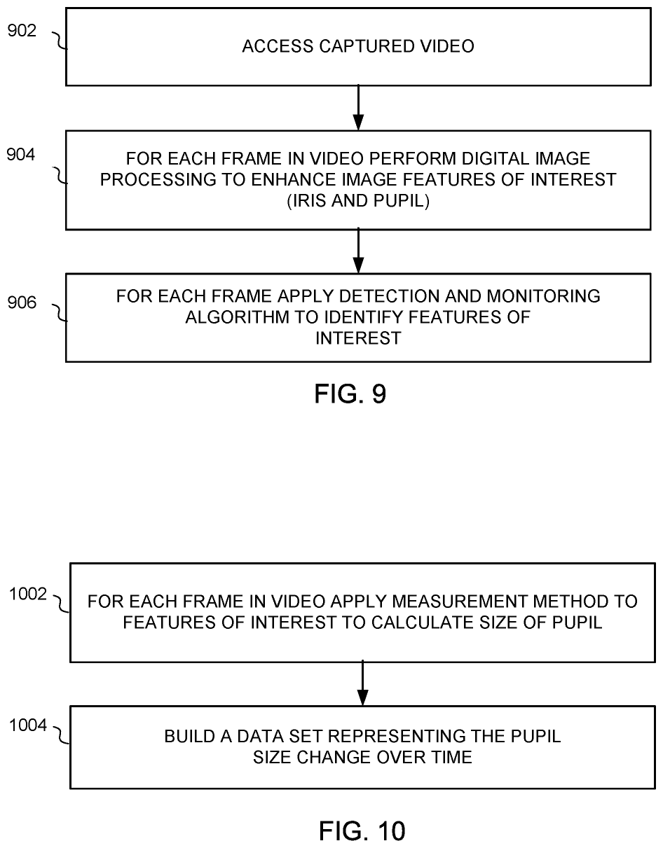

FIG. 9 is a flowchart illustrating a method to perform video processing according to one embodiment of the present disclosure. Referring to FIG. 9, the method begins with a processor, either the local processor 308 in the VCD or the remote processor 324, accessing captured video (step 902). Typically, the captured video is stored in local memory 312; however it may alternatively be stored in remote memory 326. Next, for each frame in video digital image processing is performed using the processor to enhance image features of interest, such as the iris and pupil (step 904). Finally, for each frame the processor applies a detection algorithm to identify the features of interest (step 906), after which eye feature measurements are performed.

FIG. 10 is a flowchart illustrating a method to perform eye feature(s) measurements according to one embodiment of the present disclosure. Referring to FIG. 10, the method begins with the processor for each frame in the captured and processed video applying a measurement method to features of interest to calculate size of pupil for each frame (step 1002). Next, a data set is built representing a change in pupil size over time, such that videos captured at “n” frames per second, will provide “n” consecutive measurements representing the change in pupil size over, for example, a 1 second time period (step 1004).

Generally, the above methods involve measuring in each frame of the captured video, both the size of the pupil and the iris and/or any other relevant image feature in the captured video that is not affected by the light stimuli, i.e., the iris. After features of interest are identified, a size measurement is performed by using a measurement-algorithm, which may consist of counting a number of pixels in the image including a feature of interest, and/or fitting curves like circles or ellipses, or any other geometrical shape to an area and/or perimeter of the feature of interest from which a size calculation is performed. After the measurements are performed a compensation-algorithm is used to analyze a change in size of the pupil and iris from frame to frame. Any change in size of an overall diameter of the iris is due to camera movement only, while changes in pupil size may be due to both pupil reaction to light stimuli and camera movements. The compensation-algorithm will use the change in the size of iris to extrapolate the pupil size change due only to the light stimuli, effectively removing the effect of the camera movement from the pupil size measurement and provide an accurate measurement of pupil size change due to light stimuli only. The types of movements that can affect the measurements include forward and backward motion relative to the eye, rotation around the vertical axis and tilt around the horizontal axis. The described process allows the acquisition of a PLR response signal (FIGS. 2A, 2B and 2C) that is independent from the camera movement that can be mathematical analyzed and described by calculating key descriptive parameters in the time and frequency domains.

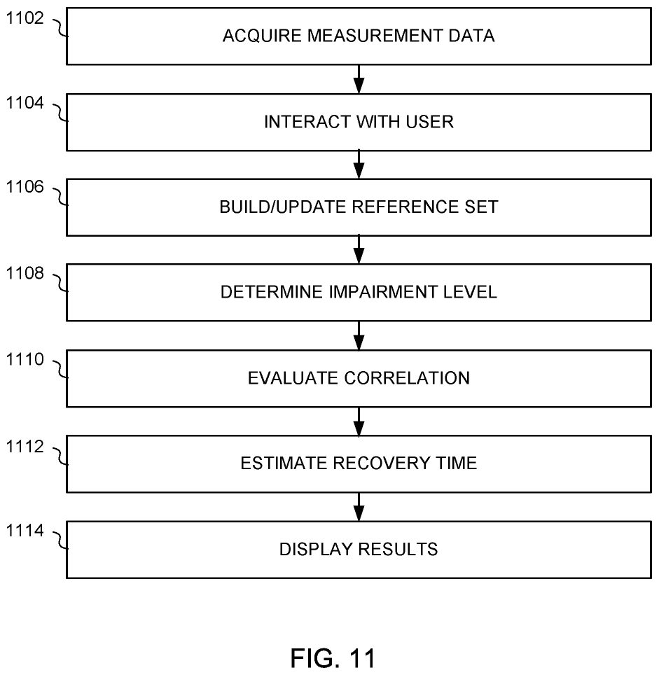

FIG. 11 is a flowchart illustrating a method to perform a correlation and prediction process according to one embodiment of the present disclosure. Referring to FIG. 11, the method begins with acquiring measurement data (step 1102). Next, the system interacts with user (step 1104). By interact with the user it is meant that the user interface may request additional information from the user, like activities performed before the test, if alcohol or any other substance has been consumed. This additional information is used to correlate the impairment level to other physical measurements. For example, in case of impairment due to alcohol consumption, a Blood Alcohol Concentration (BAC) value may be estimated. A reference set including reference parameters is built and/or updated (step 1106). Next, the level of impairment of the person undergoing test is determined by comparing calculated parameters to the reference parameters (step 1108), a significant variation of PLR is always an indication of some sort of impairment, subsequently with or without the additional information provided by the user, the correlation to other neurological impairment alteration measures is evaluated (step 1110). Where the impairment is due to intoxication, a recovery time can be estimated (step 1112) or for some other kind of impairment a recommendation of seeking immediate medical help can be provided and/or a connection with a remote medical professional can be established to interact with the User under test via audio and/or video connection to make further analysis for diagnostic, screening and/or monitoring purposes. Finally, the results are displayed to a user (step 1114).

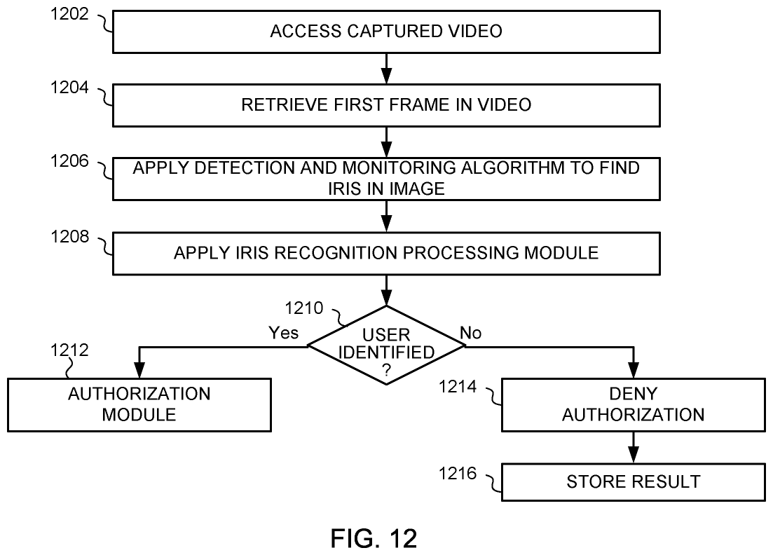

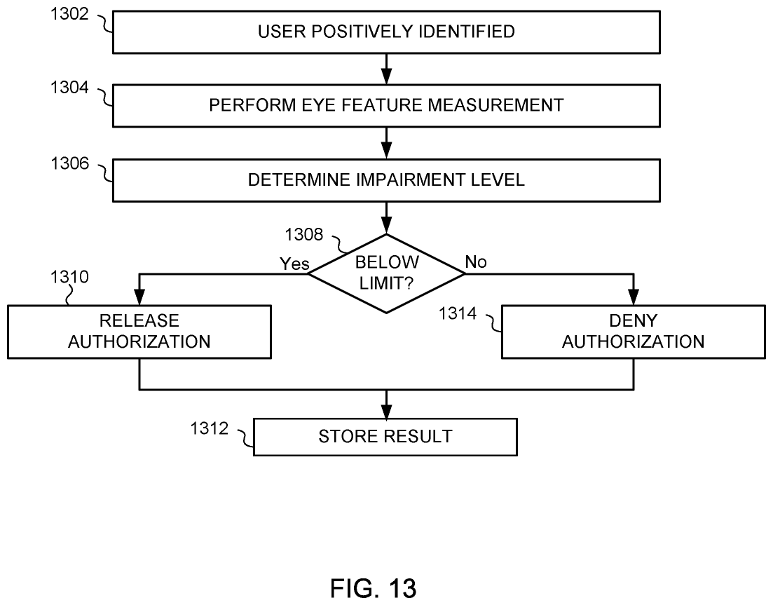

FIGS. 12 and 13 illustrate an alternative method further including software modules to perform user identification and authorization using captured image or video from the system. In particular, FIG. 12 is a flowchart illustrating a method to perform user identification through iris recognition according to an embodiment of the present disclosure. FIG. 13 is a flowchart illustrating a method to perform user authorization according to an embodiment of the present disclosure. It will be understood that the user identification or iris recognition software or module, and the authorization software or module can reside either in the local memory 312 or the remote memory 326 shown in FIG. 3.

Referring to FIG. 12, the process begins with user identification through iris recognition with access the captured video (step 1202) and retrieving the first frame in that video (step 1204). Next, the detection algorithm is applied to find an iris in the image of the first frame (step 1206). An iris recognition processing software or module is then used to compare the iris in the image of one or several frames, to a stored image of the user (step 1208). Once the user has been identified, i.e., the iris in the image of the captured frames matches a stored image of the user (step 1210), the method proceeds to the authorization software or module (step 1212) to begin testing. If the iris in the image of the first frame does not match the stored image of the user, authorization is denied (step 1214), and the result, the failed authorization attempt is stored (step 1216).

Referring to FIG. 13, once the user has been positively identified (step 1302), the eye feature measurement is performed (step 1304) and an impairment level determined (step 1306). The eye feature measurement and determination of impairment level can be accomplished using any of the systems and the methods described herein. Once the user identity has been confirmed, i.e., the iris in the video matches a stored image of the user using an iris recognition algorithm (step 1308), the method proceeds to the release authorization (step 1310) and the results of the eye feature measurement and determination of impairment level are stored (step 1312). If the user identity has been confirmed, i.e., the iris in the video does not match the stored image of the user, authorization is denied (step 1314), and the result, the failed authorization attempt.

Optionally, where the VCD is a network enabled device, such as a smartphone, the method can further include providing contact information or even automatically connect to network enabled transportation, emergency services, and/or to or a trusted personal contact previously given by the person undergoing test to render assistance, and/or to connect with a remote medical professional that will interact with the User under test via audio and/or video connection to make further analysis for diagnostic, screening, authorization and/or monitoring purposes.

Additionally, in some embodiments where the invention may be used in safety sensitive environments, where for safety and liability reasons, an employee starting a working shift is required to self-test and submit the result to an employer to receive authorization to work.

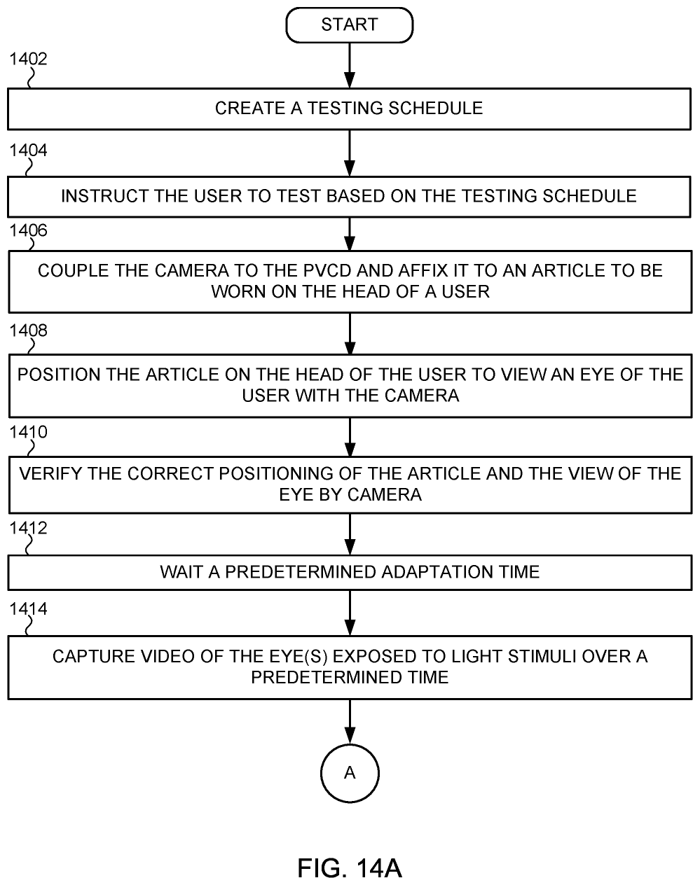

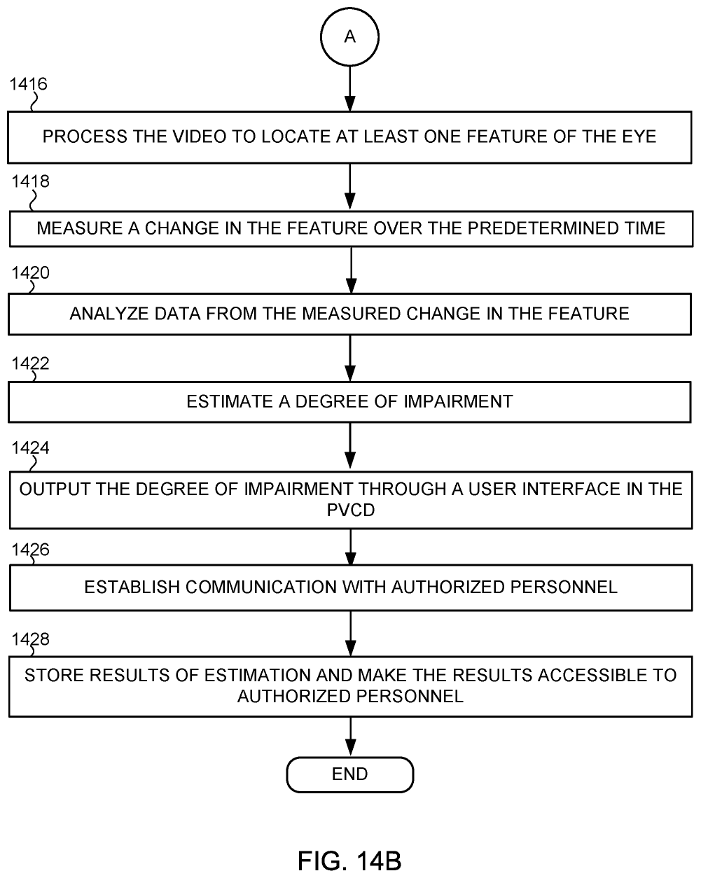

FIGS. 14A and 14B is a flowchart illustrating a method to perform a PLR response test and determine a probability and degree of impairment using any of the systems described above in connection with FIGS. 3 and 5 through 8. Although not shown in these figures, it will be understood that as described above with reference to FIG. 4 the method can, and generally does, include an initial step of receiving from the user data on the person undergoing the impairment test. User data can include a name of the person undergoing the impairment test, contact information, age, gender, height, weight, body mass, ethnicity, and other information required for the correlation and prediction step.

Referring to FIG. 14A, the method begins with the optional steps of creating a testing schedule (step 1402), and instructing a user to test for PLR alteration based on the testing schedule (step 1404). The testing schedule can be created by the user, or by an authorized person, such as an employer, family member, medical professional or officer of a court or law. The testing schedule can be randomized, scheduling tests at any time of a day, week or month with any interval of time passing between sequential tests. Alternatively, the test schedule can be regular, scheduling tests at a fixed time of day, week or month. For example, an employer may schedule a PLR alteration test with the purpose of detect on-the-job impairment to be performed at or prior to the beginning of a work shift or work week. Where the system 300/500 includes text, audio and/or video communication capability, and the step of instructing the user to test for impairment based on the testing schedule may be performed using a notification or reminder sent through the system.

Next, a camera of the system 300/500 is coupled by a wired connection or wirelessly to the VCD 302/506 and affixed to an article 502 to be worn on the head of a user (step 1406). The user then places the article 502 on their head and positions the camera and/or article to enable the camera to view at least one eye of the user (step 1408). As described above with reference to FIGS. 5 through 8 the article 502 can include glasses, safety-goggles, a helmet or protective head gear, or a frame, such as a headset.

Next, the correct positioning of the article 502 on the head of the user and the view of the eye by camera is checked and verified (step 1410). This detection and verification can be accomplished, for example, using the Smart User Interface software (SUI), as described above. After waiting or performing a countdown for a predetermined adaptation time (step 1412), the eye or eyes are exposed to light stimuli over another predetermined time using the VCD 302/506 and video or multiple images of the eye(s) captured while exposed to light stimuli over a predetermined time using the camera of the system 300/500 (step 1414). The light stimuli can arise from changes in environmental light originating from an external light source separate from the system 300/500, which are detected by the system. Alternatively, the system 300/500 further includes a light source 306 housed within the same housing 504a/504b as the camera 304 or affixed to the article 502 worn on the head of the user, and controlled by the VCD 302/506 to provide the light stimuli. The captured video is then processed to locate at least one feature of at least one of the eyes. As described above, the features of the eye located can include a pupil, an iris and/or a border between the pupil and the iris or any other discernible feature. Optionally or preferably, the video capture (step 1414) and processing of the captured video (step 1416) can include simultaneously capturing video of both eyes, and processing the video to locate at least one feature of each of the eyes. It will be understood that this embodiment enables two dual, simultaneous testing for PLR alteration, thereby improving accuracy of the test results. Additionally, this embodiment enables a comparison to be made between the PLR of each eye, either as a further indication of impairment.

Optionally, the method can further include a step (not shown in the flowchart of FIGS. 14A and 14B) of authenticating or identifying the user based on features captured in video and the method described above with reference to FIG. 12. It will be understood that an embodiment of the method using this step would be particularly desirable where the testing for impairment is being done at the request of someone other than the user.

Referring to FIG. 14B, changes in the located feature(s) of one or both eyes in response to the light stimuli over the predetermined time is measured (step 1418). Generally, this step can be accomplished as described above with reference to FIGS. 1 through 5. Data extracted from the measured change in the feature is then analyzed (step 1420). The data analysis can include calculating a number of parameters from the extracted data. Next, the calculated parameters are correlated with predetermined reference parameters in a data-store and an estimate of a degree of impairment made based on the results of the correlation (step 1422). Finally, the resultant probability and degree of impairment of the person, is output through a user interface in the VCD 302/506, such as a display and/or auditory interface to the user (step 1424). It is noted that user may be the person undergoing the impairment test or another individual.

Optionally, as shown in FIG. 14B, where the VCD 302/506 includes text, audio and/or video communication capability the method may further include the additional steps of establishing communication with authorized legal, medical or support personnel for further analysis of the estimated degree of impairment, for diagnostic, screening, authorization or monitoring purposes (step 1426), and/or where the VCD includes memory storing results of the estimated degree of impairment locally, and making the results remotely accessible to authorized personnel over a cellular or computer network (step 1428).

Thus, embodiments of to a system and method for testing for PLR alteration due to the influence of alcohol, drugs, an injury, fatigue and or neurological disorder have been described. Although the present disclosure has been described with reference to specific exemplary embodiments, it will be evident that various modifications and changes may be made to these embodiments without departing from the broader spirit and scope of the disclosure. Accordingly, the specification and drawings are to be regarded in an illustrative rather than a restrictive sense.

The Abstract of the Disclosure is provided to comply with 37 C.F.R. § 1.72(b), requiring an abstract that will allow the reader to quickly ascertain the nature of one or more embodiments of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

Reference in the description to one embodiment or an embodiment means that a particular feature, structure, or characteristic described in connection with the embodiment is included in at least one embodiment of the circuit or method. The appearances of the phrase one embodiment in various places in the specification do not necessarily all refer to the same embodiment.

What is claimed is:

1. A system comprising:
a camera in a housing affixed to an article worn on a head of a user, the housing and article operable to enable the camera to view at least one eye of the user;
a video capture device (VCD) coupled to the camera to receive images or video therefrom, the VCD including a user interface, a processor, and a memory; and
a software program stored in the memory and executed by the processor, the software program including:
a video capture module to continuously or repeatedly capture images or video of the eye using the camera;
local correlation and prediction module to:
locate a feature of the eye;
performing a Pupillary Light Reflex (PLR) response test and measure a change in the feature relative to a stored image, or over a predetermined time in response to light stimuli or changes in ambient light;
extract data from the measured change in the feature;
analyze the PLR response signal in the time and frequency domains to extract key descriptive parameters of the signal
calculate parameters from the extracted data; and
correlate the calculated parameters with predetermined reference parameters and predict a degree of impairment based on results of the correlation; and
a user interface module to output a probability and degree of impairment to the user and/or to a third party monitor.

2. The system of claim 1 wherein the camera and VCD are operable to continuously or repeatedly capture images or video of the eye to automatically monitor impairment to the user either continuously or intermittently.

3. The system of claim 1 wherein the article comprises glasses, safety-goggles, a frame of a face-shield, a head-band, a helmet or a hard or soft hat worn by the user.

4. The system of claim 1 wherein the VCD is coupled to the camera through a wireless or a wired connection or is integrated with the camera.

5. The system of claim 1 wherein the light stimuli comprise environmental light provided by a light source external to the system.

6. The system of claim 1 further comprising a light source affixed to the article worn on the head of the user, and controlled by the VCD to provide the light stimuli.

7. The system of claim 1 wherein the feature of the eye comprises an iris, and wherein the local correlation and prediction module further comprises computer program code to authenticate an identity of the user by applying an iris recognition algorithm to the captured images or video of the eye.

8. The system of claim 7 wherein the software program further comprises computer program code to transmit the identity of the user and a probability and degree of impairment to a remote server.

9. The system of claim 1 wherein the VCD further includes a network interface device through which the VCD is coupled to a remote memory, and wherein the software program may be at least partially stored in the remote memory.

10. A system comprising:
a camera affixed to a surface facing a head of a user, the camera positioned to view at least one eye of the user;
a video capture device (VCD) coupled to the camera to receive images or video therefrom, the VCD including a user interface, a processor, and a memory; and
a software program stored in the memory and executed by the processor, the software program including:
a video capture module to continuously or repeatedly capture images or video of the eye using the camera;
local correlation and prediction module to:
locate a feature of the eye;
performing a Pupillary Light Reflex (PLR) response test and measure a change in the feature relative to a stored image, or over a predetermined time in response to light stimuli;
extract data from the measured change in the feature;
analyze the PLR response signal in the time and frequency domains to extract key descriptive parameters of the signal;
calculate parameters from the extracted data; and
correlate the calculated parameters with predetermined reference parameters and predict a degree of impairment based on results of the correlation; and
a user interface module to output a probability and degree of impairment to the user and/or to a third party monitor.

11. The system of claim 10 wherein the camera and the VCD are operable to continuously or repeatedly capture images or video of the eye to automatically monitor impairment to the user either continuously or intermittently.

12. The system of claim 10 wherein the surface comprises a surface of a machine being operated by the user.

13. The system of claim 10 wherein the surface comprise an instrument panel of a vehicle being operated by the user.

14. The system of claim 10 wherein the surface comprise a mirror of a vehicle being operated by the user.

15. The system of claim 10 wherein the surface is substantially transparent to at least some wavelengths of light to which the camera is sensitive, and wherein the camera is affixed to an opposite side of the surface from the user.

16. The system of claim 10 wherein the VCD is a portable video capture device (PVCD).

17. The system of claim 16 wherein the PVCD is coupled to the camera through a wireless or a wired connection or is integrated with the camera.

18. The system of claim 10 wherein the light stimuli comprises environmental light provided by a light source external to the system.

19. The system of claim 10 further comprising a light source affixed to the surface and controlled by the VCD to provide the light stimuli.

20. The system of claim 10 wherein the feature of the eye comprises an iris, and wherein the local correlation and prediction module further comprises computer program code to authenticate an identity of the user by applying an iris recognition algorithm to the captured images or video of the eye.

21. The system of claim 20 wherein the software program further comprises computer program code to transmit the identity of the user and a probability and degree of impairment to a remote server.

22. The system of claim 10 wherein the VCD further includes a network interface device through which the VCD is coupled to a remote memory, and wherein the software program may be at least partially stored in the remote memory.

23. A method comprising:
capturing video of at least one eye of a user with a camera in a housing affixed to an article worn on a head of the user;
transmitting video from the camera to a video capture device (VCD) coupled thereto;
processing the video to locate at least one feature of the eye;
performing a Pupillary Light Reflex (PLR) response test and measuring a change in the feature relative to a stored image, or over a predetermined time in response to light stimuli;
analyzing data from the measured change in the feature, wherein analyzing data includes extracting data from the measured change in the feature, analyze the PLR response signal in the time and frequency domains to extract key descriptive parameters of the signal;
calculating parameters from the extracted data, correlating the calculated parameters against predetermined reference parameters and predicting a degree of impairment based on results of the correlation; and
outputting through a user interface in the VCD a probability and degree of impairment to the user and/or to a third party monitor.

24. The method of claim 23 wherein the capturing video, transmitting video, processing the video, measuring a change in the feature and analyzing data from the measured change are done automatically either continuously or intermittently to continuously monitor impairment of the user.

25. The method of claim 23 wherein transmitting video is done through a wireless or a wired connection.

26. The method of claim 23 further comprising controlling with the VCD a light source within the housing to provide the light stimuli.

27. The method of claim 23 wherein the feature of the eye comprises an iris, and wherein processing the video further comprises authenticating an identity of the user by applying an iris recognition algorithm to the captured video of the eye.

28. The method of claim 27 further comprising transmitting the identity of the user and a probability and degree of impairment to a remote server.
